(19) United States
(12) Patent Application Publication
(10) Pub. No.: US 2005/0266011 A1
(43) Pub. Date: Dec. 1, 2005

(54) Method and formulation for transdermal delivery of immunologically active agents

(76) Inventors: Yuh-Fun Maa, Millbrae, CA (US); Mahmoud Ameri, Fremont, CA (US); Scott Sellers, San Mateo, CA (US)

Correspondence Address: Ralph C. Francis;Francis Law Group, 1942 Embarcadero, Oakland, CA, 94606, US

(21) Appl. No.: 11/112,311

(22) Filed: Apr. 21, 2005

Related U.S. Application Data
(60) Provisional application No. 60/572,861, filed on May 19, 2004.

(51) Int. Cl. A61K 39/00

(52) U.S. Cl. 424/184.1

(57) ABSTRACT

A method for formulating an immunologically active agent and an apparatus for delivery same, the method comprising the steps of providing a bulk immunologically active agent, subjecting the bulk immunologically active agent to tangential-flow filtration to provide an immunologically active agent solution, adding at least one excipient to the agent solution and spray-drying the agent solution to form an immunologically active agent product; the apparatus comprising a microprojection member that includes a plurality of microprojections having a biocompatible coating disposed thereon that includes a spray-dried immunologically active agent. In a preferred embodiment, the immunologically active agent comprises an influenza vaccine, more preferably, a split-varion influenza vaccine.

### CROSS-REFERENCE TO RELATED APPLICATIONS

This application claims the benefit of U.S. Provisional Application No. 60/572,861, filed May 19, 2004.

### FIELD OF THE PRESENT INVENTION

The present invention relates generally to immunologically active agent compositions and methods forming and delivering such compositions. More particularly, the invention relates to methods and formulations for transdermal delivery of spray-dried immunologically active agents, particularly, influenza vaccine.

### BACKGROUND OF THE INVENTION

Active agents, such as vaccines, are most conventionally administered either orally or by injection. Unfortunately, many active agents are completely ineffective or have radically reduced efficacy when orally administered, since they either are not absorbed or are adversely affected before entering the bloodstream and thus do not possess the desired activity. On the other hand, the direct injection of the agent into the bloodstream, while assuring no modification of the agent during administration, is a difficult, inconvenient, painful and uncomfortable procedure which sometimes results in poor patient compliance.

Transdermal delivery is thus a viable alternative for administering active agents, particularly, vaccines that would otherwise need to be delivered via hypodermic injection or intravenous infusion. The word “transdermal”, as used herein, is generic term that refers to delivery of an active agent (e.g., a therapeutic agent, such as a drug or an immunologically active agent, such as a vaccine) through the skin to the local tissue or systemic circulatory system without substantial cutting or penetration of the skin, such as cutting with a surgical knife or piercing the skin with a hypodermic needle. Transdermal agent delivery includes delivery via passive diffusion as well as delivery based upon external energy sources, such as electricity (e.g., iontophoresis) and ultrasound (e.g., phonophoresis).

Passive transdermal agent delivery systems, which are more common, typically include a drug reservoir that contains a high concentration of an active agent. The reservoir is adapted to contact the skin, which enables the agent to diffuse through the skin and into the body tissues or bloodstream of a patient.

As is well known in the art, the transdermal drug flux is dependent upon the condition of the skin, the size and physical/chemical properties of the drug molecule, and the concentration gradient across the skin. Because of the low permeability of the skin to many drugs, transdermal delivery has had limited applications. This low permeability is attributed primarily to the stratum corneum, the outermost skin layer which consists of flat, dead cells filled with keratin fibers (i.e., keratinocytes) surrounded by lipid bilayers. This highly-ordered structure of the lipid bilayers confers a relatively impermeable character to the stratum corneum.

One common method of increasing the passive transdermal diffusional flux involves mechanically penetrating the outermost skin layer(s) to create micropathways in the skin. There have been many techniques and devices developed to create pathways into the skin. Illustrative is the drug delivery device disclosed in U.S. Pat. No. 3,964,482.

Other systems and apparatus that employ tiny skin piercing elements to enhance transdermal agent delivery are disclosed in U.S. Pat. Nos. 5,879,326, 3,814,097, 5,250,023, 3,964,482, Reissue No. 25,637, and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365; all incorporated herein by reference in their entirety.

The disclosed systems and apparatus employ piercing elements of various shapes and sizes to pierce the outermost layer (i.e., the stratum corneum) of the skin. The piercing elements disclosed in these references generally extend perpendicularly from a thin, flat member, such as a pad or sheet. The piercing elements in some of these devices are extremely small, some having a microprojection length of only about 25-400 microns and a microprojection thickness of only about 5-50 microns. These tiny piercing/cutting elements make correspondingly small microslits/microcuts in the stratum corneum for enhancing transdermal agent delivery therethrough.

The disclosed systems further typically include a reservoir for holding the agent and also a delivery system to transfer the agent from the reservoir through the stratum corneum, such as by hollow tines of the device itself. One example of such a device is disclosed in WO 93/17754, which has a liquid agent reservoir. The reservoir must, however, be pressurized to force the liquid agent through the tiny tubular elements and into the skin.

As disclosed in U.S. patent application Ser. No. 10/045,842, which is fully incorporated by reference herein, it is also possible to have the active agent that is to be delivered coated on the microprojections instead of contained in a physical reservoir. This eliminates the necessity of a separate physical reservoir and developing an agent formulation or composition specifically for the reservoir.

As is well known in the art, the agent formulation and method of coating the formulation on the microprojections are critical factors in transdermal delivery via coated microprojections. Indeed, if a vaccine is employed in the agent formulation that is unstable or does not have sufficient shelf-life, the vaccine may not, and in many instances, will not have the desired (or required) effectiveness.

As is also well known in the art, biological materials, such as vaccines, are often dried to stabilize them for storage or distribution. However, drying often causes a reduction in efficacy and/or activity. Freeze-drying or lyophilization has been found to significantly reduce such damage, and can obviate the need for refrigerated storage.

Lyophilization is the process of removing water from a product by sublimation and desorption. For pharmaceutical compounds that undergo hydrolytic degradation, lyophilization offers a means of improving stability and shelf life.

A typical lyophilization system includes a drying chamber with temperature controlled shelves, a condenser to trap water removed from the product, a cooling system to supply refrigerant to the shelves and condenser, a vacuum system to reduce the pressure in the chamber and a condenser to facilitate the drying process. Many active agents, such as vaccines, proteins, peptides, and antibiotics, have been successfully lyophilized.

Many microorganisms and proteins can be subjected to lyophilization, without adverse side effects. Lyophilization is thus a favored method of drying vaccines, pharmaceuticals, blood fractions, and diagnostics. For example, U.S. Pat. No. 3,991,179 discloses that influenza vaccine may be freeze-dried and reconstituted while remaining immunologically active.

Co-pending U.S. patent application Ser. No. 11/084,631, filed Apr. 1, 2004, similarly discloses a pre-formulation process for an influenza vaccine that includes freeze-drying. The noted process also provides a highly concentrated vaccine formulation as an intermediate product.

Lyophilized materials typically reconstitute easily and quickly because of the porous structure remaining after the ice has sublimed. Upon rehydration, the stabilized materials can be easily formulated for transdermal delivery, either as a coating on microprojections or inclusion in an agent formulation in a reservoir.

A typical lyophilization cycle consist of three phases: (i) freezing, (ii) primary drying and (iii) secondary drying. Conditions in the dryer are varied throughout the cycle to insure that the resulting product has the desired physical and chemical properties, and that the required stability is achieved.

There are, however, several drawbacks and disadvantages associated with a lyophilization process. For example, the total amount of material that can be subjected to lyophilization at one time is limited and the entire process can take several days. Thus, despite its advantages, lyophilization is a very complex, expensive and time-consuming process.

It would therefore be desirable to provide a method for formulating a stable immunologically active agent, and in particular, an influenza vaccine, that is more economical than lyophilization while maintaining sufficient activity and minimizing damage.

It is therefore an object of the present invention to provide a stabilized immunologically active agent formulation that retains sufficient activity to be immunologically or biologically effective.

It is another object of the present invention to provide a method of stabilizing immunologically active agents that minimizes manufacturing time and costs.

It is another object of the present invention to provide a stabilized influenza vaccine that can be readily administered transdermally in an immunologically (or biologically) effective amount.

It is yet another object of the present invention to impart specific particle characteristics in a stabilized immunologically active agent.

### SUMMARY OF THE INVENTION

In accordance with the above objects and those that will be mentioned and will become apparent below, in one embodiment of the invention, the method for formulating an immunologically active agent comprises the following steps: (i) providing a bulk immunologically active agent, (ii) subjecting the bulk immunologically active agent to tangential-flow filtration to provide an immunologically active agent solution, (iii) adding at least one excipient to the agent solution, and (iv) spray-drying the agent solution to form an immunologically active agent product.

Preferably, the immunologically active agent solution is spray-dried at an inlet temperature in the range of approximately 60° C. to about 250° C., and more preferably, in the range of approximately 100° C. to about 200° C.

Also preferably, the immunologically active agent solution is spray-dried at a feed rate in the range from approximately 0.5 mL/min to 30 mL/min, and more preferably, in the range from approximately 2 mL/min to 10 mL/min.

In another aspect of the invention, the immunologically active agent retains at least a 12-month room temperature stability following spray-drying.

In accordance with yet another aspect of the invention, the immunologically active agent retains a potency of at least approximately 70%, and more preferably, at least approximately 80%.

In a preferred embodiment of the invention, the immunologically active agent comprises an influenza vaccine. More preferably, the immunologically active agent comprises a split-varion influenza vaccine. Even more preferably, the immunologically active agent comprises a hemagglutinin.

In alternative embodiments of the invention, the immunologically active agent comprises an antigenic agent or vaccine selected from the group consisting of viruses and bacteria, protein-based vaccines, polysaccharide-based vaccine, and nucleic acid-based vaccines.

Suitable immunologically active agents include, without limitation, antigens in the form of proteins, polysaccharide conjugates, oligosaccharides, and lipoproteins. These subunit vaccines include Bordetella pertussis (recombinant PT vaccine—acellular), Clostridium tetani (purified, recombinant), Corynebacterium diptheriae (purified, recombinant), Cytomegalovirus (glycoprotein subunit), Group A streptococcus (glycoprotein subunit, glycoconjugate Group A polysaccharide with tetanus toxoid, M protein/peptides linked to toxin subunit carriers, M protein, multivalent type-specific epitopes, cysteine protease, C5a peptidase), Hepatitis B virus (recombinant Pre-bS1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial surface protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), and Vibrio cholerae (conjugate lipopolysaccharide).

Whole virus or bacteria include, without limitation, weakened or killed viruses, such as cytomegalo virus, hepatitis B virus, hepatitis C virus, human papillomavirus, rubella virus, and varicella zoster, weakened or killed bacteria, such as bordetella pertussis, clostridium tetani, corynebacterium diptheriae, group A streptococcus, legionella pneumophila, neisseria meningitis, pseudomonas aeruginosa, streptococcus pneumoniae, treponema pallidum, and vibrio cholerae, and mixtures thereof.

A number of commercially available vaccines, which contain antigenic agents also have utility with the present invention, include, without limitation, flu vaccines, Lyme disease vaccine, rabies vaccine, measles vaccine, mumps vaccine, chicken pox vaccine, small pox vaccine, hepatitis vaccine, pertussis vaccine, and diphtheria vaccine.

Vaccines comprising nucleic acids that can also be delivered according to the methods of the invention include, without limitation, single-stranded and double-stranded nucleic acids, such as, for example, supercoiled plasmid DNA; linear plasmid DNA; cosmids; bacterial artificial chromosomes (BACs); yeast artificial chromosomes (YACs); mammalian artificial chromosomes; and RNA molecules, such as, for example, mRNA.

Suitable immune response augmenting adjuvants which, together with the vaccine antigen, can comprise the vaccine include, without limitation, aluminum phosphate gel; aluminum hydroxide; algal glucan: β-glucan; cholera toxin B subunit; CRL1005: ABA block polymer with mean values of x=8 and y=205; gamma insulin: linear (unbranched) β-D(2->1) polyfructofuranoxyl-α-D-glucose; Gerbu adjuvant: N-acetylglucosamine-(β 1-4)-N-acetylmuramyl-L-alanyl-D-glutamine (GMDP), dimethyl dioctadecylammonium chloride (DDA), zinc L-proline salt complex (Zn-Pro-8); Imiquimod (1-(2-methypropyl)-1H-imidazo[4,5-c]quinolin-4-amine; ImmTher™: N-acetylglucoaminyl-N-acetylmuramyl-L-Ala-D-isoGlu-L-Ala-glycerol dipalmitate; MTP-PE liposomes: C₅₉H₁₀₈N₆O₁₉PNa-3H₂0 (MTP); Murametide: Nac-Mur-L-Ala-D-Gln-OCH₃; Pleuran: β-glucan; QS-21; S-28463: 4-amino-a,a-dimethyl-1H-imidazo[4,5-c]quinoline-1-ethanol; salvo peptide: VQGEESNDK.HCl (IL-1β 163-171 peptide); and threonyl-MDP (Termurtide™): N-acetyl muramyl-L-threonyl-D-isoglutamine, and interleukine 18, IL-2 IL-12, IL-15. Adjuvants also include DNA oligonucleotides, such as, for example, CpG containing oligonucleotides. In addition, nucleic acid sequences encoding for immuno-regulatory lymphokines such as IL-18, IL-2 IL-12, IL-15, IL-4, IL 10, gamma interferon, and NF kappa B regulatory signaling proteins can be used.

Suitable excipients include, without limitation, pharmaceutical grades of carbohydrates, including monosaccharides, disaccharides, cyclodextrins, and polysaccharides; starch; cellulose; salts (e.g., sodium or calcium phosphates, calcium sulfate, magnesium sulfate); citric acid; tartaric acid; glycine; low, medium or high molecular weight polyethylene glycols (PEG's); pluronics; surfactants; and combinations thereof. Preferred excipients comprise disaccharides and polysaccharides.

In accordance with another aspect of the invention, the immunologically active agent solution further comprises a stabilizing agent selected from the group consisting of non-reducing sugars, polysaccharides, reducing sugars and cyclodextrins.

Suitable non-reducing sugars for use in the methods and compositions of the invention include, for example, sucrose, trehalose, stachyose, or raffinose.

Suitable polysaccharides for use in the methods and compositions of the invention include, for example, dextran, soluble starch, dextrin, and insulin.

Suitable reducing sugars for use in the methods and compositions of the invention include, for example, monosaccharides, such as, for example, apiose, arabinose, lyxose, ribose, xylose, digitoxose, fucose, quercitol, quinovose, rhamnose, allose, altrose, fructose, galactose, glucose, gulose, hamamelose, idose, mannose, tagatose, and the like; and disaccharides such as, for example, primeverose, vicianose, rutinose, scillabiose, cellobiose, gentiobiose, lactose, lactulose, maltose, melibiose, sophorose, and turanose, and the like.

Suitable cyclodextrins for use in the methods and compositions of the invention include, for example, Alpha-cyclodextrin, Beta-cyclodextrin, Gamma-cyclodextrin, glucosyl-alpha-cyclodextrin, maltosyl-alpha-cyclodextrin, glucosyl-beta-cyclodextrin, maltosyl-beta-cyclodextrin, hydroxypropyl beta-cyclodextrin, 2-hydroxypropyl-beta-cyclodextrin, 2-hydroxypropyl-gamma-cyclodextrin, hydroxyethyl-beta-cyclodextrin, methyl-beta-cyclodextrin, sulfobutylether-alpha-cyclodextrin, sulfobutylether-beta-cyclodextrin, and sulfobutylether-gamma-cyclodextrin. Most preferred solubilising/complexing agents are beta-cyclodextrin, hydroxypropyl beta-cyclodextrin, 2-hydroxypropyl-beta-cyclodextrin and sulfobutylether7 beta-cyclodextrin.

In accordance with a further embodiment of the invention, the apparatus for transdermally delivering an immunologically active agent comprises a microprojection member that includes a plurality of microprojections that are adapted to pierce through the stratum corneum into the underlying epidermis layer, or epidermis and dermis layers, the microprojection member having a biocompatible coating disposed thereon that includes a spray-dried immunologically active agent. In a preferred embodiment, the immunologically active agent comprises an influenza vaccine, more preferably, a split-varion influenza vaccine.

In accordance with another embodiment of the invention, the apparatus for transdermally delivering an immunologically active agent comprises a microprojection member that includes a plurality of microprojections and a reservoir adapted to receive an agent formulation, the agent formulation including a spray-dried immunologically active agent. In a preferred embodiment, the immunologically active agent comprises an influenza vaccine, more preferably, a split-varion influenza vaccine.

In accordance with one embodiment of the invention, the method for delivering an immunologically active agent comprises the following steps: (i) providing a microprojection member having a plurality of microprojections, (ii) providing a bulk immunologically active agent, (iii) subjecting the bulk immunologically active agent to tangential-flow filtration to provide a first immunologically active agent solution, (iv) adding at least one excipient (e.g., sucrose) to the first agent solution, (v) spray-drying the first agent solution to form a vaccine product, (vi) reconstituting the vaccine product with a first solution (e.g., water) to form a second immunologically active agent solution, (vii) forming a biocompatible coating that includes the second immunologically active agent solution, (viii) coating the microprojection member with the biocompatible coating, and (viii) applying the coated microprojection member to the skin of a subject.

In accordance with yet another embodiment of the invention, the method for delivering an immunologically active agent comprises the following steps: (i) providing a transdermal delivery device, the delivery device including a microprojection member having a plurality of microprojections and a reservoir adapted to receive an agent formulation, (ii) providing a bulk immunologically active agent, (iii) subjecting the bulk immunologically active agent to tangential-flow filtration to provide a first immunologically active agent solution, (iv) adding at least one excipient (e.g., sucrose) to the first agent solution, (v) spray-drying the first agent solution to form a vaccine product, (vi) reconstituting the vaccine product with a first solution (e.g., water) to form a second immunologically active agent solution, (vii) forming an agent formulation that includes the second immunologically active agent solution, (viii) loading the reservoir with the agent formulation and (ix) applying the coated microprojection member to the skin of a subject.

In a preferred embodiment of the invention, the immunologically active agent comprises hemagglutinin and the step of applying the microprojection member to the skin of the subject delivers approximately 45 μg of hemagglutinin. More preferably, at least approximately 50% of the immunologically active agent is delivered to the APC-abundant epidermal layer.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is an illustration of an influenza virus particle;

FIG. 2 is a flow chart of one embodiment of the formulation process for an immunologically active agent, according to the invention;

FIGS. 3 and 4 are SEM images illustrating the morphology of stabilized influenza vaccines, according to the invention;

FIG. 5 is a bar graph illustrating the potencies of various stabilized influenza vaccines, according to the invention;

FIG. 6 is a graphical illustration comparing the molecular weights of various stabilized influenza vaccines, according to the invention; and

FIG. 7 is a graphical illustration comparing activities of stabilized influenza vaccines with lyophilized vaccines, according to the invention.

### DETAILED DESCRIPTION OF THE INVENTION

Before describing the present invention in detail, it is to be understood that this invention is not limited to particularly exemplified materials, formulations, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “an immunologically active agent” includes two or more such agents; reference to “a microprojection” includes two or more such microprojections and the like.

### Definitions

The term “transdermal”, as used herein, means the delivery of an agent into and/or through the skin for local or systemic therapy.

The term “transdermal flux”, as used herein, means the rate of transdermal delivery.

The term “co-delivering”, as used herein, means that a supplemental agent(s) is administered transdermally either before the agent is delivered, before and during transdermal flux of the agent, during transdermal flux of the agent, during and after transdermal flux of the agent, and/or after transdermal flux of the agent. Additionally, two or more immunologically active agents may be formulated in the biocompatible coatings of the invention, resulting in co-delivery of different immunologically active agents.

The term “biologically active agent”, as used herein, refers to a composition of matter or mixture containing an active agent or drug, which is pharmacologically effective when administered in a therapeutically effective amount. Examples of such active agents include, without limitation, small molecular weight compounds, polypeptides, proteins, oligonucleotides, nucleic acids and polysaccharides.

The term “immunologically active agent”, as used herein, refers to a composition of matter or mixture containing an antigenic agent and/or a “vaccine” from any and all sources, which is capable of triggering a beneficial immune response when administered in an immunologically effective amount. A specific example of an immunologically active agent is an influenza vaccine.

Further examples of immunologically active agents include, without limitation, viruses and bacteria, protein-based vaccines, polysaccharide-based vaccine, and nucleic acid-based vaccines.

Suitable immunologically active agents include, without limitation, antigens in the form of proteins, polysaccharide conjugates, oligosaccharides, and lipoproteins. These subunit vaccines include Bordetella pertussis (recombinant PT vaccine—acellular), Clostridium tetani (purified, recombinant), Corynebacterium diptheriae (purified, recombinant), Cytomegalovirus (glycoprotein subunit), Group A streptococcus (glycoprotein subunit, glycoconjugate Group A polysaccharide with tetanus toxoid, M protein/peptides linked to toxin subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial surface protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), and Vibrio cholerae (conjugate lipopolysaccharide).

Whole virus or bacteria include, without limitation, weakened or killed viruses, such as cytomegalo virus, hepatitis B virus, hepatitis C virus, human papillomavirus, rubella virus, and varicella zoster, weakened or killed bacteria, such as bordetella pertussis, clostridium tetani, corynebacterium diptheriae, group A streptococcus, legionella pneumophila, neisseria meningitis, pseudomonas aeruginosa, streptococcus pneumoniae, treponema pallidum, and vibrio cholerae, and mixtures thereof.

A number of commercially available vaccines, which contain antigenic agents also have utility with the present invention, include, without limitation, flu vaccines, Lyme disease vaccine, rabies vaccine, measles vaccine, mumps vaccine, chicken pox vaccine, small pox vaccine, hepatitis vaccine, pertussis vaccine, and diphtheria vaccine.

Vaccines comprising nucleic acids that can also be delivered according to the methods of the invention include, without limitation, single-stranded and double-stranded nucleic acids, such as, for example, supercoiled plasmid DNA; linear plasmid DNA; cosmids; bacterial artificial chromosomes (BACs); yeast artificial chromosomes (YACs); mammalian artificial chromosomes; and RNA molecules, such as, for example, mRNA. The size of the nucleic acid can be up to thousands of kilobases. The nucleic acid can also be coupled with a proteinaceous agent or can include one or more chemical modifications, such as, for example, phosphorothioate moieties.

Suitable immune response augmenting adjuvants which, together with the vaccine antigen, can comprise the vaccine include, without limitation, aluminum phosphate gel; aluminum hydroxide; algal glucan: β-glucan; cholera toxin B subunit; CRL1005: ABA block polymer with mean values of x=8 and y=205; gamma insulin: linear (unbranched) β-D(2->1) polyfructofuranoxyl-α-D-glucose; Gerbu adjuvant: N-acetylglucosamine-(β 1-4)-N-acetylmuramyl-L-alanyl-D-glutamine (GMDP), dimethyl dioctadecylammonium chloride (DDA), zinc L-proline salt complex (Zn-Pro-8); Imiquimod (1-(2-methypropyl)-1H-imidazo[4,5-c]quinolin-4-amine; ImmTher™: N-acetylglucoaminyl-N-acetylmuramyl-L-Ala-D-isoGlu-L-Ala-glycerol dipalmitate; MTP-PE liposomes: C₅₉H₁₀₈N₆O₁₉PNa-3H₂0 (MTP); Murametide: Nac-Mur-L-Ala-D-Gln-OCH₃; Pleuran: β-glucan; QS-21; S-28463: 4-amino-a,a-dimethyl-1H-imidazo[4,5-c]quinoline-1-ethanol; salvo peptide: VQGEESNDK.HCl (IL-1β 163-171 peptide); and threonyl-MDP (Termurtide™): N-acetyl muramyl-L-threonyl-D-isoglutamine, and interleukine 18, IL-2 IL-12, IL-15. Adjuvants also include DNA oligonucleotides, such as, for example, CpG containing oligonucleotides. In addition, nucleic acid sequences encoding for immuno-regulatory lymphokines such as IL-18, IL-2 IL-12, IL-15, IL-4, IL10, gamma interferon, and NF kappa B regulatory signaling proteins can be used.

The term “excipient”, as used herein, refers to pharmaceutical grades of carbohydrates including, without limitation, monosaccharides, disaccharides, cyclodextrins, and polysaccharides (e.g., dextrose, sucrose, lactose, raffinose, mannitol, sorbitol, inositol, dextrins, and maltodextrins); starch; cellulose; salts (e.g., sodium or calcium phosphates, calcium sulfate, magnesium sulfate); citric acid; tartaric acid; glycine; low, medium or high molecular weight polyethylene glycols (PEG's); pluronics; surfactants; and combinations thereof.

The term “biologically effective amount” or “biologically effective rate”, as used herein, refers to the amount or rate of the immunologically active agent needed to stimulate or initiate the desired immunologic, often beneficial result. The amount of the immunologically active agent employed in the coatings of the invention will be that amount necessary to deliver an amount of the immunologically active agent needed to achieve the desired immunological result. In practice, this will vary widely depending upon the particular immunologically active agent being delivered, the site of delivery, and the dissolution and release kinetics for delivery of the immunologically active agent into skin tissues.

As will be appreciated by one having ordinary skill in the art, the dose of the immunologically active agent that is delivered can also be varied or manipulated by altering the microprojection array (or patch) size, density, etc.

The term “coating formulation”, as used herein, is meant to mean and include a freely flowing composition or mixture that is employed to coat the microprojections and/or arrays thereof.

The term “biocompatible coating” and “solid coating”, as used herein, is meant to mean and include a “coating formulation” in a substantially solid state.

The term “microprojections”, as used herein, refers to piercing elements which are adapted to pierce or cut through the stratum corneum into the underlying epidermis layer, or epidermis and dermis layers, of the skin of a living animal, particularly a mammal and more particularly a human.

The term “microprojection member”, as used herein, generally connotes a microprojection array comprising a plurality of microprojections arranged in an array for piercing the stratum corneum. The microprojection member can be formed by etching or punching a plurality of microprojections from a thin sheet and folding or bending the microprojections out of the plane of the sheet to form a configuration. The microprojection member can also be formed in other known manners, such as by forming one or more strips having microprojections along an edge of each of the strip(s) as disclosed in U.S. Pat. No. 6,050,988, which is hereby incorporated by reference in its entirety.

Microprojection members that can be employed with the present invention include, but are not limited to, the members disclosed in U.S. Pat. Nos. 6,083,196, 6,050,988 and 6,091,975, and U.S. Pat. Pub. No. 2002/0016562, which are incorporated by reference herein in their entirety.

As indicated above, the present invention comprises an apparatus, method and formulation for transdermal delivery of an immunologically active agent. In one embodiment of the invention, the apparatus includes a microprojection member (or system) having a plurality of microprojections (or array thereof) that are adapted to pierce through the stratum corneum into the underlying epidermis layer, or epidermis and dermis layers, the microprojection member having a biocompatible coating disposed thereon that includes at least one spray-dried immunologically active agent.

In a preferred embodiment of the invention, the immunologically active agent comprises an influenza vaccine, more preferably, a spray-dried, split-varion influenza vaccine. According to the invention, upon piercing the stratum corneum layer of the skin, the biocompatible coating is dissolved by body fluid (intracellular fluids and extracellular fluids such as interstitial fluid) and the influenza vaccine is released into the skin (i.e., bolus delivery) for systemic therapy.

According to the invention, the kinetics of the coating dissolution and release will depend on many factors, including the nature of the immunologically active agent, the coating process, the coating thickness and the coating composition (e.g., the presence of coating formulation additives). Depending on the release kinetics profile, it may be necessary to maintain the coated microprojections in piercing relation with the skin for extended periods of time. This can be accomplished by anchoring the microprojection member to the skin using adhesives or by using anchored microprojections, such as described in WO 97/48440, which is incorporated by reference herein in its entirety.

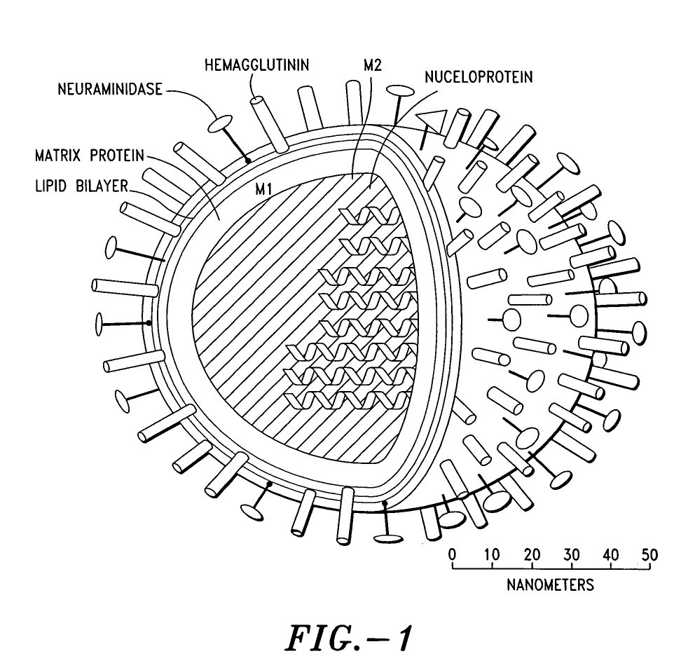

As is well known in the art, the influenza virus particle consists of many protein components with hemagglutinin (HA) as the primary surface antigen responsible for the induction of protective anti-HA antibodies in humans. An illustration of an influenza particle is shown in FIG. 1.

Immunologically, influenza A viruses are classified into subtypes on the basis of two surface antigens: HA and neuraminidase (NA). HA is the protein responsible for the ability of the flu virus to agglutinate red blood cells and for the binding of the virus to cells via its attachment to sialic acid. HA is now recognized as the major virulence factor associated with this virus. Immunity to these antigens, especially to the hemagglutinin, reduces the likelihood of infection and lessens the severity of the disease if infection occurs.

The antigenic characteristics of circulating strains provide the basis for selecting the virus strains included in each year's vaccine. Every year, the influenza vaccine contains three virus strains (usually two type A and one B) that represent the influenza viruses that are likely to circulate worldwide in the coming winter. Influenza A and B can be distinguished by differences in their nucleoproteins and matrix proteins. Type A is the most common strain and is responsible for the major human pandemics. The HA content of each strain in the trivalent vaccine is typically set at 15 μg for a single human dose, i.e., 45 μg total HA.

A split-varion or split-antigen vaccine is preferred for use in the practice of the invention. Since an incomplete portion of the virus is used, the risk of infection is essentially eliminated.

One means of producing a split-varion vaccine is to propagate the influenza virus in chicken embryos and then harvest the virus-containing fluids and inactivate them with formaldehyde. The influenza virus is concentrated and purified in a linear sucrose density gradient solution using a continuous flow centrifuge. The virus is then chemically disrupted using Polyethylene Glycol p-Isooctylphenyl Ether (Triton® X-100, Rohm and Haas, Co.) to produce a split-varion. The split-varion is then further purified by chemical means and suspended in sodium phosphate-buffered isotonic sodium chloride solution.

By virtue of the unique formulation process, discussed in detail below, a full human dose of the influenza vaccine, i.e., 45 μg of hemagglutinin, can be transdermally delivered to the APC-abundant epidermal layer, the most immuno-competent component of the skin, via a coated microprojection array, wherein at least 50% of the influenza vaccine is delivered to the noted epidermal layer. More importantly, the antigen remains immunogenic in the skin to elicit strong antibody and sero-protective immune responses. Further, the dry coated vaccine formulation can maintain at least a 12-month room temperature stability.

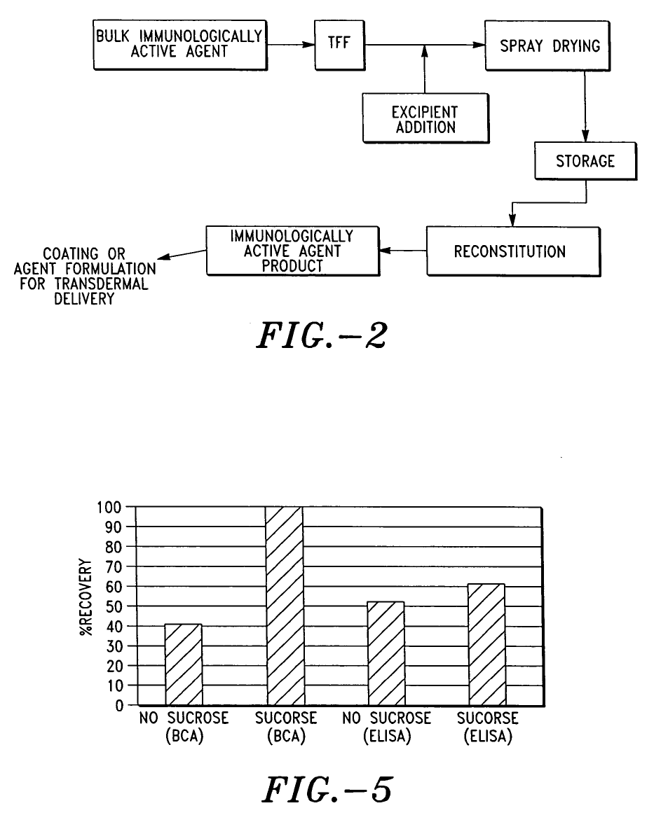

Referring now to FIG. 2, there is shown a flow diagram of one embodiment of the formulation process of the invention. As illustrated in FIG. 2, the formulation process includes the steps of tangential-flow filtration (TFF), spray-drying and reconstitution.

Upon receipt of the vaccine, the first step is to subject the vaccine to tangential-flow filtration. As is well known in the art, tangential-flow filtration is typically employed to remove low molecular weight materials.

Following TFF, the vaccine is preferably formulated with a lyoprotective excipient, such as sucrose or trehalose, and spray-dried.

As is well known in the art, spray drying is the transformation of a material into a dried particulate powder by spraying a liquid solution of the material into a hot drying medium. Spray drying can form a powdered spherical product directly from a solution or dispersion.

The main advantages of spray drying are rapid drying and minimal temperature increase of the material during the spray-drying process. Further, the methods are suited for the continuous production of dry solids in either powder, granulate or agglomerate form from liquids as solutions, emulsions and suspensions. Spray-drying also provides an end-product having precise quality standards regarding particle size and particle size distribution, residual moisture content, particle density, particle morphology, and other characteristics.

A typical spray dryer apparatus includes a feed pump, atomizer, air heater, air dispenser and drying chamber. The apparatus further includes systems for exhaust air cleaning and powder recovery.

Generally, the spray drying process comprises the atomization of a liquid feedstock into a spray of droplets and contacting the droplets with hot air in a drying chamber. The sprays are produced by either rotary (wheel) or nozzle atomizers. Evaporation of moisture from the droplets and formation of dry particles is performed under controlled temperature and airflow conditions. In most operations, the powder is discharged continuously from the drying chamber.

In accordance with the present invention, a fine mist of solubilized material (e.g., immunologically active agent solution) is introduced into a large conical chamber where it comes into contact with air that has been heated to about 100° C. or more, depending on the material or agent being dried. For the immunologically active agents of the invention, the spray-drying is preferably conducted at an inlet temperature in the range of approximately 60° C. to 250° C., more preferably, in the range of approximately 100° C. to 200° C. Suitable feed rates are in the range of approximately 0.5 mL/min to 30 mL/min, more preferably, in the range of approximately 2 L/min to 10 mL/min.

Typically, the drying air and particles move through the drying chamber in the same direction. The product temperature on discharge from the dryer is generally lower than the exhaust air temperature and, hence, provides an ideal mode for drying heat sensitive products.

When operating with a rotary atomizer, the air disperser creates a high degree of air rotation, which provides a uniform temperature throughout the drying chamber. Alternatively, a non-rotating airflow can be used with nozzle atomizers.

According to the invention, various air flow configurations can be employed to tailor the process to the agent being dried and the desired result. For example, counter flow conditions with drying air and particles moving through the drying chamber in opposite directions generally provides a degree of heat treatment during drying. The temperature of the powder discharged from the dryer is also usually higher than the exhaust air temperature.

Another type of air flow is mixed flow with particle movement through the drying chamber both with and against the air flow. This mode is suitable for heat stable products where coarse powder requirements necessitate the use of nozzle atomizers, spraying upwards into an incoming airflow, or for heat sensitive products where the atomizer sprays droplets downward towards an integrated fluid bed and wherein the air inlet and outlet are located at the top of the drying chamber.

According to the invention, the noted formulation process provides highly stable, concentrated and solid-state hemagglutinin (HA) formulations as intermediate products. The intermediate products are also highly potent and immunologenic.

Without being limited to any particular theory, the presence of non-hemagglutinin components, including the chemical disrupter, lipids, lipid-protein complexes and other proteins, enhance the stability of the spray-dried vaccine.

As will be appreciated by one have ordinary skill in the art, the noted formulation process of the invention can be modified and adapted to formulate various vaccine source materials and forms thereof. For example, the process could be adapted to use raw materials received at higher concentrations. In this case, the diafiltration step would not be necessary and the high concentration raw materials would be directly spray-dried and reconstituted to produce the coating formulation.

The formulation process could also be modified for use with high purity raw materials, such as, but not limited to, cell derived influenza vaccines. In this case, the materials may be of sufficient purity that the TFF and reconstitution steps would be unnecessary.

According to the invention, a multitude of immunologically active agents or vaccines can be subjected to the formulation process of the invention to provide highly stable vaccine formulations. In a preferred embodiment of the invention, the immunologically active agent comprises an influenza vaccine, more preferably, a split-varion influenza vaccine.

The immunologically active agent can additionally comprise viruses and bacteria, protein-based vaccines, polysaccharide-based vaccines, and nucleic acid-based vaccines. Suitable antigenic agents include, without limitation, antigens in the form of proteins, polysaccharide conjugates, oligosaccharides, and lipoproteins. These subunit vaccines include Bordetella pertussis (recombinant PT vaccine—acellular), Clostridium tetani (purified, recombinant), Corynebacterium diptheriae (purified, recombinant), Cytomegalovirus (glycoprotein subunit), Group A streptococcus (glycoprotein subunit, glycoconjugate Group A polysaccharide with tetanus toxoid, M protein/peptides linked to toxin subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial surface protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), and Vibrio cholerae (conjugate lipopolysaccharide).

Whole virus or bacteria include, without limitation, weakened or killed viruses, such as cytomegalo virus, hepatitis B virus, hepatitis C virus, human papillomavirus, rubella virus, and varicella zoster, weakened or killed bacteria, such as bordetella pertussis, clostridium tetani, corynebacterium diptheriae, group A streptococcus, legionella pneumophila, neisseria meningitis, pseudomonas aeruginosa, streptococcus pneumoniae, treponema pallidum, and vibrio cholerae, and mixtures thereof.

Additional commercially available vaccines, which contain antigenic agents, include, without limitation, flu vaccines, Lyme disease vaccine, rabies vaccine, measles vaccine, mumps vaccine, rubella vaccine, pertussis vaccine, tetanus vaccine, typhoid vaccine, rhinovirus vaccine, hemophilus influenza B, polio vaccine, pneumococal vaccine, meningococcal vaccine, RSU vaccine, herpes vaccine, HIV vaccine, chicken pox vaccine, small pox vaccine, hepatitis vaccine (including types A,B and D) and diphtheria vaccine.

Vaccines comprising nucleic acids include, without limitation, single-stranded and double-stranded nucleic acids, such as, for example, supercoiled plasmid DNA; linear plasmid DNA; cosmids; bacterial artificial chromosomes (BACs); yeast artificial chromosomes (YACs); mammalian artificial chromosomes; and RNA molecules, such as, for example, mRNA. The size of the nucleic acid can be up to thousands of kilobases. In addition, in certain embodiments of the invention, the nucleic acid can be coupled with a proteinaceous agent or can include one or more chemical modifications, such as, for example, phosphorothioate moieties.

Suitable immune response augmenting adjuvants which, together with the vaccine antigen, can comprise the vaccine include, without limitation, aluminum phosphate gel; aluminum hydroxide; algal glucan: β-glucan; cholera toxin B subunit; CRL1005: ABA block polymer with mean values of x=8 and y=205; gamma insulin: linear (unbranched) β-D(2->1) polyfructofuranoxyl-α-D-glucose; Gerbu adjuvant: N-acetylglucosamine-(β 1-4)-N-acetylmuramyl-L-alanyl-D-glutamine (GMDP), dimethyl dioctadecylammonium chloride (DDA), zinc L-proline salt complex (Zn-Pro-8); Imiquimod (1-(2-methypropyl)-1H-imidazo[4,5-c]quinolin-4-amine; ImmTher™: N-acetylglucoaminyl-N-acetylmuramyl-L-Ala-D-isoGlu-L-Ala-glycerol dipalmitate; MTP-PE liposomes: C₅₉H₁₀₈N₆O₁₉PNa-3H₂0 (MTP); Murametide: Nac-Mur-L-Ala-D-Gln-OCH₃; Pleuran: β-glucan; QS-21; S-28463: 4-amino-a, a-dimethyl-1H-imidazo[4,5-c]quinoline-1-ethanol; salvo peptide: VQGEESNDK.HCl (IL-1β 163-171 peptide); and threonyl-MDP (Termurtide™): N-acetyl muramyl-L-threonyl-D-isoglutamine, and interleukine 18, IL-2 IL-12, IL-15. Adjuvants also include DNA oligonucleotides, such as, for example, CpG containing oligonucleotides. In addition, nucleic acid sequences encoding for immuno-regulatory lymphokines such as IL-18, IL-2 IL-12, IL-15, IL-4, IL10, gamma interferon, and NF kappa B regulatory signaling proteins can be used.

In a preferred embodiment of the invention, the vaccine formulation includes at least one excipient. Suitable excipients include, without limitation, pharmaceutical grades of carbohydrates including monosaccharides, disaccharides, cyclodextrins, and polysaccharides (e.g., dextrose, sucrose, lactose, raffinose, mannitol, sorbitol, inositol, dextrins, and maltodextrins); starch; cellulose; salts (e.g., sodium or calcium phosphates, calcium sulfate, magnesium sulfate); citric acid; tartaric acid; glycine; low, medium or high molecular weight polyethylene glycols (PEG's); pluronics; surfactants; and combinations thereof. Preferably, the excipient comprises disaccharides and polysaccharides.

According to the invention, the preferred excipients help maintain the potency of the vaccine and the recovery of the antigen during reconstitution. The amount of excipient employed depends upon the immunologically active agent. For example, in one embodiment, the agent to excipient ratio is preferably in the range of approximately 2:1 to 1:20 for influenza vaccines, more preferably, approximately 1:4.

According to the invention, the spray-dried immunologically active agents of the invention can be readily employed in the coating and hydrogel formulations, and methods of and apparatus for transdermally delivering same, described in detail in Co-Pending U.S. patent application Ser. No. 11/084,631, filed Apr. 1, 2004, and U.S. patent application Ser. No. 11/084,635, filed Apr. 13, 2004, which are expressly incorporated herein in their entirety.

As set forth in the noted Co-Pending applications, one method that can be employed to coat the microprojections or arrays thereof comprises dip-coating. Dip-coating generally comprises partially or totally immersing the microprojections into a coating solution. By use of a partial immersion technique, it is possible to limit the coating to only the tips of the microprojections.

A further coating method comprises roller coating, which employs a roller coating mechanism that similarly limits the coating to the tips of the microprojections. The roller coating method is disclosed in U.S. application Ser. No. 10/099,604 (Pub. No. 2002/0132054), which is incorporated by reference herein in its entirety.

As will be appreciated by one having ordinary skill in the art, vaccine formulations containing a spray-dried immunologically active agent of the invention can also be employed in conjunction with a wide variety of iontophoresis and electrotransport systems. Illustrative are the electrotransport systems disclosed in U.S. Pat. Nos. 5,147,296, 5,080,646, 5,169,382 and 5,169,383, which are incorporated herein in their entirety.

### EXAMPLES

The following studies and examples illustrate the formulations, methods and processes of the invention. The examples are for illustrative purposes only and are not meant to limit the scope of the invention in any way.

### Example 1

As is known in the art, tangential-flow filtration (TFF) allows diafiltration and concentration to be performed at the same time. A TFF system (Millipore, Labscale) equipped with a Pellicon XL, regenerated cellulose membrane (Millipore, 50 cm², 30 kD MWCO) was thus employed for the diafiltration and concentration of the vaccine raw material. The volume of the vaccine solution was reduced to 1/20^{th}- 1/50^{th} of the original volume, increasing the HA concentration to 5-10 mg HA/mL. A buffer solution was also added for buffer exchange and concentration.

In a first study, an influenza vaccine, a monovalent A/Panama strain (Fluzone® from Aventis Pasteur) was diafiltered and concentrated, as described above, to about 10 mg HA/mL. 5 mL of this concentrated A/Panama solution was spray dried directly, without any additional excipients (Formulation A). In another formulation, 50 mg of sucrose was added to 5 mL of the A/Panama concentrate (Formulation B). The formulations were spray dried using a Yamato Laboratory Spray Dryer.

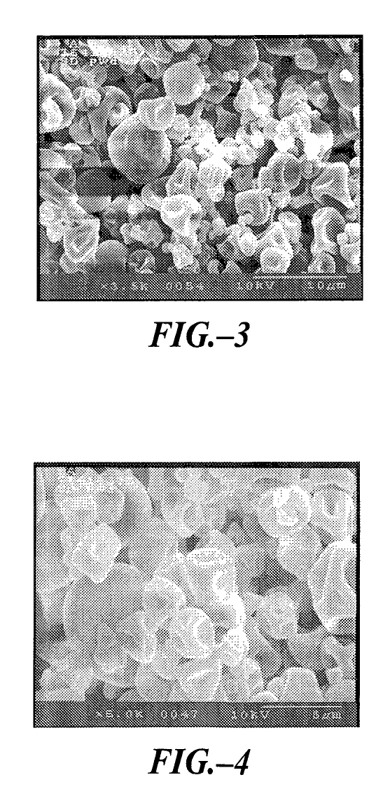

Formulation A was spray dried at an inlet temperature of 120° C., an outlet temperature of 120° C. and a liquid feed rate of 2 ml/min. This produced 47.1 mg of powder representing a 31% yield and was designated Sample 1. The morphology of Sample 1 is shown in FIG. 3.

Formulation B was spray dried at an inlet temperature of 140° C., an outlet temperature of 105° C. and a liquid feed rate of 2 ml/min. This produced 55.48 mg of powder representing a 26% yield and was designated Sample 2. The morphology of Sample 2 is shown in FIG. 4.

Both powder formulations were reconstituted to a concentration of about 1.2 mg HA/mL with water. The sucrose-containing formulation exhibited slight precipitation while the sucrose-free formulation had significantly more precipitation. The formulations were assayed for protein and potency using bicinchoninic acid (BCA) analysis and enzyme-linked immunosorbent assay (ELISA).

Sample 2 demonstrated a BCA HA concentration of 1.34±0.11 and an ELISA HA concentration of 0.83±0.04. Sample 1 demonstrated a BCA HA concentration of 0.55±0.03 and an ELISA HA concentration of 0.72±0.03. The relative recovery of HA in these assays, as compared to the theoretical HA concentration of 1.2 mg HA/mL, is shown in FIG. 5. As illustrated in FIG. 5, there was no protein loss in the sucrose-containing formulation.

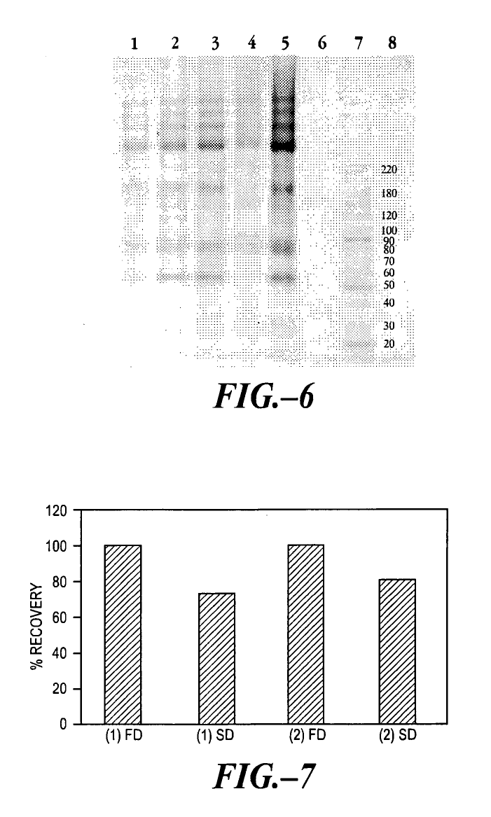

Referring now to FIG. 6, there is shown the results of a sodium dodecyl sulfate polyacrylamide gel electrophoresis (SDS-PAGE) analysis of the molecular weights of the formulations and various reagents. Specifically, Lane 1 was loaded with the A/Panama L/N FA 108621 vaccine with 35 μL at 180 μgHA/mL, which corresponds to 6.3 μg HA. Lane 2 was loaded with the TFF concentrate non-sucrose formulation (Formulation A) prior to spray-drying with 10 μL at about 1 mgHA/mL, which corresponds to 10 μg HA. Lane 3 was loaded with the TFF concentrate sucrose formulation (Formulation B) prior to spray-drying with 10 μL at about 1 mgHA/mL, which corresponds to 10 μg HA. Lane 4 was loaded with 20 μL of spray-dried non-sucrose formulation (Formulation A) at 8 mg Powder/mL. Lane 5 was loaded with 20 μL of spray-dried sucrose formulation (Formulation B) at 10 mg Powder/mL. Lanes 6 and 8 were loaded with buffer blank and Lane 7 was loaded with a standard molecular weight marker. These results indicate that there were no changes in molecular weight species for the sucrose-containing spray-dried formulation.

### Example 2

In a further study, two formulations were prepared using a monovalent B/Victoria strain of hemagglutinin. Formulation C comprised antigen and sucrose in a 1:4 weight ratio. Formulation D comprised antigen, trehalose and mannitol in a 1:2:2 weight ratio. Both formulations were spray-dried (SD) and freeze dried (FD) and then subjected to BCA protein analysis and SRID (single radio-immuno diffusion) potency analysis.

The BCA assay of the SD and FD formulations demonstrated that both methods of stabilization resulted in full recovery of the hemagglutinin antigen. As shown in FIG. 7, SRID analysis demonstrates that spray-drying provides potency retention of approximately 70% for Formulation C and approximately 80% for Formulation D. The results thus demonstrate that spray-drying is a viable means for stabilizing immunologically active agents, while offering great economy and efficiency with respect to lyophilization.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

1. A method for formulating an immunologically active agent comprising the steps of:
providing a bulk immunologically active agent,
subjecting said bulk immunologically active agent to tangential-flow filtration to provide an immunologically active agent solution,
adding at least one excipient to said immunologically active agent solution, and
spray-drying said immunologically active agent solution to form an immunologically active agent product.

2. The method of claim 1, wherein the step of spray-drying said immunologically active agent solution is conducted at an inlet temperature in the range of approximately 60° C. to about 250° C.

3. The method of claim 2, wherein the step of spray-drying said immunologically active agent solution is conducted at an inlet temperature in the range of approximately 100° C. to about 200° C.

4. The method of claim 1, wherein the step of spray-drying said immunologically active agent solution is conducted at a feed rate in the range from approximately 0.5 mL/min to 30 mL/min.

5. The method of claim 4, wherein the step of spray-drying said immunologically active agent solution is conducted at a feed rate in the range from approximately 2 mL/min to 10 mL/min.

6. The method of claim 1, wherein said immunologically active agent retains at least a 12-month room temperature stability.

7. The method of claim 1, wherein said immunologically active agent retains a potency of at least approximately 70%.

8. The method of claim 9, wherein said immunologically active agent retains a potency of at least approximately 80%.

9. The method of claim 1, wherein said immunologically active agent comprises an influenza vaccine.

10. The method of claim 9, wherein said immunologically active agent comprises a split-varion influenza vaccine.

11. The method of claim 9, wherein said immunologically active agent comprises hemagglutinin.

12. The method of claim 1, wherein said immunologically active agent comprises an antigenic agent selected from the group consisting of viruses, bacteria, protein-based vaccines, polysaccharide-based vaccines, and nucleic acid-based vaccines.

13. The method of claim 1, wherein said immunologically active agent comprises an antigen selected from the group consisting of proteins, polysaccharide conjugates, oligosaccharides, and lipoproteins.

14. The method of claim 1, wherein said immunologically active agent is selected from the group consisting of Bordetella pertussis (recombinant PT vaccine—acellular), Clostridium tetani (purified, recombinant), Corynebacterium diptheriae (purified, recombinant), Cytomegalovirus (glycoprotein subunit), Group A streptococcus (glycoprotein subunit, glycoconjugate Group A polysaccharide with tetanus toxoid, M protein/peptides linked to toxin subunit carriers, M protein, multivalent type-specific epitopes, cysteine protease, C5a peptidase), Hepatitis B virus (recombinant Pre-bS1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial surface protein), Neisseria meningitides (glycoconjugate with tetanus toxoid), Pseudomonas aeruginosa (synthetic peptides), Rubella virus (synthetic peptide), Streptococcus pneumoniae (glyconconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), and Vibrio cholerae (conjugate lipopolysaccharide).

15. The method of claim 1, wherein the immunologically active agent is selected from the group consisting of flu vaccines, Lyme disease vaccine, rabies vaccine, measles vaccine, mumps vaccine, chicken pox vaccine, small pox vaccine, hepatitis vaccine, pertussis vaccine, and diphtheria vaccine.

16. The method of claim 1, further comprising the step of adding an immune response augmenting adjuvant to said immunologically active agent solution.

17. The method of claim 16, wherein said immune response augmenting adjuvant is selected from the group consisting of aluminum phosphate gel, aluminum hydroxide, algal glucan: β-glucan, cholera toxin B subunit, CRL1005: ABA block polymer with mean values of x=8 and y=205, gamma insulin: linear (unbranched) β-D(2->1) polyfructofuranoxyl-α-D-glucose, Gerbu adjuvant: N-acetylglucosamine-(β1-4)-N-acetylmuramyl-L-alanyl-D-glutamine (GMDP), dimethyl dioctadecylammonium chloride (DDA), zinc L-proline salt complex (Zn-Pro-8), Imiquimod (1-(2-methypropyl)-1 H-imidazo[4,5-c]quinolin-4-amine, ImmTher™: N-acetylglucoaminyl-N-acetylmuramyl-L-Ala-D-isoGlu-L-Ala-glycerol dipalmitate, MTP-PE liposomes: C₅₉H₁₀₈N₆O₁₉PNa-3H₂0 (MTP), Murametide: Nac-Mur-L-Ala-D-Gln-OCH₃, Pleuran: β-glucan, QS-21, S-28463: 4-amino-a,a-dimethyl-1H-imidazo[4,5-c]quinoline-1-ethanol, salvo peptide: VQGEESNDK.HCl (IL-11, 163-171 peptide), and threonyl-MDP (Termurtide™): N-acetyl muramyl-L-threonyl-D-isoglutamine, interleukine-18, interleukine-2, interleukine-12, interleukine-15, DNA oligonucleotides, CpG containing oligonucleotides, nucleic acid sequences encoding for immuno-regulatory lymphokines, gamma interferon, and NF kappa B regulatory signaling proteins.

18. The method of claim 1, wherein said excipient is selected from the group consisting of carbohydrates, monosaccharides, disaccharides, cyclodextrins, polysaccharides, starch, cellulose, salts, sodium phosphates, calcium phosphates, calcium sulfate, magnesium sulfate, citric acid, tartaric acid, glycine, polyethylene glycols (PEG's), pluronics, and surfactants.

19. The method of claim 1, wherein said immunologically active agent solution further comprises a stabilizing agent selected from the group consisting of non-reducing sugars, polysaccharides, reducing sugars, and cyclodextrins.

20. An apparatus for transdermally delivering an immunologically active agent comprising a microprojection member having a plurality of stratum corneum-piercing microprojections, wherein said microprojection member has a biocompatible coating disposed thereon including a spray-dried immunologically active agent.

21. The apparatus of claim 20, wherein said immunologically active agent comprises an influenza vaccine.

22. The apparatus of claim 13, wherein said immunologically active agent comprises a split-varion influenza vaccine.

23. An apparatus for transdermally delivering an immunologically active agent comprising a microprojection member having a plurality of stratum corneum-piercing microprojections, and a reservoir adapted to receive said agent formulation, the agent formulation including a spray-dried immunologically active agent.

24. The apparatus of claim 23, wherein said immunologically active agent comprises an influenza vaccine.

25. The apparatus of claim 23, wherein said immunologically active agent comprises a split-varion influenza vaccine.

26. A method for delivering an immunologically active agent comprising the steps of:
providing a microprojection member having a plurality of microprojections,
providing a bulk immunologically active agent,
subjecting said bulk immunologically active agent to tangential-flow filtration to provide a first immunologically active agent solution,
adding at least one excipient to said first agent solution,
spray-drying said first agent solution to form a vaccine product,
reconstituting said vaccine product with a first solution to form a second immunologically active agent solution,
applying said second immunologically active agent solution to said microprojection member, and
applying the coated microprojection member to the skin of a subject.

27. The method of claim 26, further comprising the step of forming a biocompatible coating including said second immunologically active agent solution, and wherein the step of applying said second immunologically active agent solution to said microprojection member comprises coating said microprojection member with said biocompatible coating

28. The method of claim 26, wherein said microprojection member further comprises a reservoir, further comprising the step of forming an agent formulation that includes said second immunologically active agent solution, and wherein the step of applying said second immunologically active agent solution to said microprojection member comprises loading said reservoir with said agent formulation.

29. The method of claim 26, wherein said immunologically active agent comprises an influenza vaccine.

30. The method of claim 29, wherein said immunologically active agent comprises a split-varion influenza vaccine.

31. The method of claim 29, wherein the step of applying the coated microprojection member to the skin of a subject delivers approximately 45 μg of said immunologically active agent.

32. The method of claim 26, wherein the step of applying the coated microprojection member to the skin of a subject delivers at least approximately 50% of said immunologically active agent to the APC-abundant epidermal layer.
